(19) United States
(12) Patent Application Publication
(10) Pub. No.: US 2010/0307859 A1
(43) Pub. Date: Dec. 9, 2010

(54) ACOUSTIC DEVICE FOR LINEAR PERCEIVED-SOUND ATTENUATION

(75) Inventors: Franck Lopez, Castelnau Le Lez (FR); Pricille Angot, Sommieres (FR)

Correspondence Address: THE NATH LAW GROUP, 112 South West Street, Alexandria, VA, 22314, US

(73) Assignee: Earsonics, Clapiers (FR)

(21) Appl. No.: 12/809,128

(22) PCT Filed: Dec. 19, 2008
(86) PCT No.: PCT/FR2008/052382

§ 371 Date: Jun. 18, 2010

(30) Foreign Application Priority Data
Dec. 21, 2007 (FR) 0760183
Apr. 14, 2008 (FR) 0852492

(51) Int. Cl. G10K 11/16; H04R 25/00; A61F 11/08

(52) U.S. Cl. 181/129; 181/135; 181/192

(57) ABSTRACT

The present invention relates to an acoustic device for linear perceived-sound attenuation, comprising a channel along which the sound propagates from an entry to an exit, characterized in that it comprises, combined so as to make the perceived sound undergo a linear attenuation, at its entry, means for attenuating and setting into resonance sound propagating through the latter as far as at least one filter opening as exit, onto means for accentuating the filtered sound, in particular its short-wave frequencies, the accentuation means consisting of a frustoconical portion of the channel having a cross-section that increases from the housing towards the exit.

### BACKGROUND OF THE INVENTION

(1) Field of the Invention

This invention relates to the field of hearing, in particular hearing protection.

The object of the invention is an acoustic attenuation device, of the noise filter type.

Such a device will find an application in particular as a hearing aid made to measure, but also within the framework of earplugs.

More specifically, this invention aims at a linear perceived-sound attenuation, contrarily to plugs and filters attenuating sound selectively, at the level of one or more portions of the hearing spectrum only. The invention claims to be able to render attenuated the entire acoustic spectrum, in particular the high frequencies corresponding to the middle and high pitch.

(2) Description of the Prior Art

The state of the art already uses hearing aid filters having a linear perceived-sound attenuation. However, the existing filters do not permit a linear attenuation over the whole range of frequencies of the acoustic spectrum. In particular, the audible frequencies above 8000 Hz are strongly attenuated after filtration, generating an abrupt drop over this interval of the spectrum beyond this limit. In the worst-case scenario, there might be a clipping causing a complete disappearance of the frequencies beyond said limit.

Besides, another disadvantage of the existing filtration devices resides in that they do in no way take into account any psycho-acoustic criteria of interpretation of tones. Whereas acoustics studies the nature and properties of sound waves, psycho-acoustics deals with the way they are picked up by the auditory system and how they are interpreted by the brain. Based on this study, it is inferred that the perception of the characteristics of a sound has no objective measuring values.

In particular, the clipping of the high frequencies destroys the even harmonics situated between 10 and 16 kHz, the latter contributing to a pleasant sonority or a so-called <<musical>> sound.

### SUMMARY OF THE INVENTION

The aim of this invention is to cope with the disadvantages of the state of the art by providing an acoustic device for linear sound attenuation over a wide spectrum band. The invention claims to be able to control the level of attenuation and preserve a linear response after filtration for a better listening comfort.

In particular, the invention offers the possibility of attenuating a sound without any loss of harmonics and high frequencies. The invention produces an attenuated filtered sound comparable to a <<loudness>> effect, i.e. a frequency correction permitting to subjectively obtain a more powerful sound during a low-volume listening.

In order to illustrate the difference between the existing devices and the device according to the invention, we refer to FIG. A. It shows the attenuation in decibels (DB) depending on the acoustic spectrum, expressed in Hertz (Hz).

The lowest curve, designated by G, corresponds to the attenuation of a plug, for example made of foam. The curves D, E and F transcribe the non-linear attenuation of the known devices. A drop similar to the attenuation of a plug is observed. Even if the curve D, which corresponds to a plug made to measure by molding, goes back up after 4000 Hz, a complete loss of the most high-pitched frequencies beyond 8000 Hz is observed.

An example of such a non-linear attenuation device is described in EP 0 112 594, in which a molded hearing aid receives a non linear filtration device. The latter includes a channel along which the sound propagates from an entry to an exit, the first portion of said channel being removable, so as to permit the insertion of a filter at the level of the end of its second portion.

Because of the small diameter of the channel, a cavity can be provided so as to avoid the obstruction by dust or fat particles. However, this cavity can provoke uncontrolled acoustic interferences.

Besides, the small section of said channel considerably attenuates the acoustic spectrum in the medium frequency and completely clips the high frequencies, beyond 8000 kHz.

The curve C corresponds to a linear filtration device as mentioned above. One observes that the curve is on average rising up to 4 kHz, then declining.

To the contrary, the curves A and B, representing the attenuation of two embodiments of filtration device according to the invention, are reversed, generating the so-called <<loudness>> acoustic effect, pleasant to the ear because such a curve respects the tonal balance of the human ear.

To this end, this invention combines opposite technologies consisting in, on the one hand, linearly attenuating the perceived sound and, on the other hand, obtaining a resonance of the latter. Furthermore, the sound is amplified upon exiting the device according to the invention, through a projection phenomenon in order to accentuate short-wave frequencies, i.e. the middle and high pitch.

Thus, the invention permits to linearly attenuate a perceived sound, without losing the sonority and the quality of the latter, rendering the high frequencies, in particular beyond 8 kHz. Therefore, a <<loudness>> attenuation effect is achieved, which is more natural to the ear, through a stronger attenuation in the more sensitive low frequencies and by enhancing the high frequencies, offering a linear perception.

Moreover, a particular implementation within the framework of hearing aids molded to measure permits to considerably improve audio quality, obtaining a more natural and musical sound.

Therefore, the object of the invention is an acoustic device for linear perceived-sound attenuation, comprising a channel along which said sound propagates from an entry to an exit, wherein it comprises, combined so as to make the perceived sound undergo a linear attenuation, at its entry, means for attenuating and setting into resonance said sound propagating through the latter to at least one filter opening, at its exit, onto means for accentuating the filtered sound, in particular its short-wave frequencies, said means for accentuating consisting of a frustoconical portion of said channel having a cross-section that increases from said housing toward said exit.

The invention also relates to an earplug, comprising such an acoustic device.

The invention also relates to a hearing aid, made by molding to measure and formed so as to receive, and receiving said acoustic device.

Further features and advantages of the invention will become more evident from the following detailed description of the non-restrictive embodiments of the invention, referring to the attached figures.

### BRIEF DESCRIPTION OF THE DRAWING

FIG. A is a comparative representation of the attenuation curves of the state-of-the-art devices with respect to the invention.

FIG. 1 represents a perspective view of an acoustic device according to an embodiment of the invention;

FIG. 2 represents a cross-sectional view of the device of FIG. 1;

FIG. 3 represents a cross-sectional view of the device of FIG. 1 according to another embodiment.

FIG. 4 represents a perspective view of an acoustic device according to another embodiment of the invention; and

FIG. 5 represents a cross-sectional view of the device of FIG. 4.

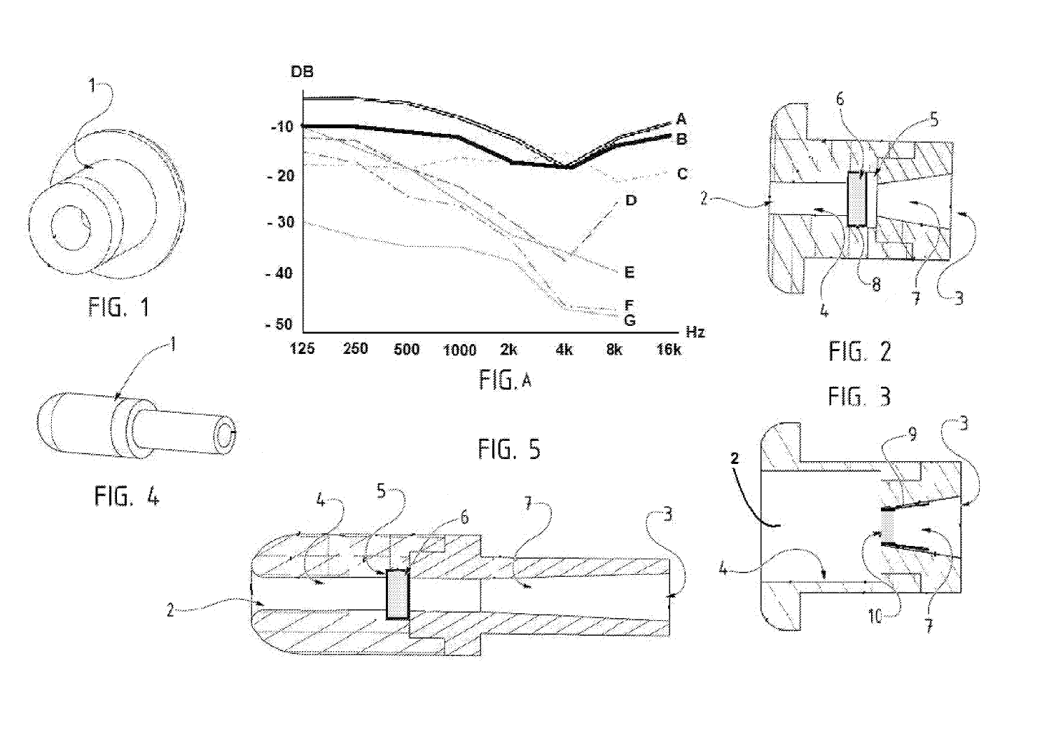

This invention relates to an acoustic device for linear perceived-sound attenuation.

Preferably, but not restrictively, according to the embodiments shown in the drawings, such a device has a general cylindrical shape.

### DETAILED DESCRIPTION OF THE PREFERRED EMBODIMENTS

It includes a channel 1 provided for inside said device, passing through the latter longitudinally. Said channel 1 propagates the sound from an entry 2 to an exit 3 situated at each one of the ends of said channel 1, opposite to each other.

Throughout said channel, the invention provides means capable of attenuating, setting into resonance, filtering and accentuating said sound, and the combination of which permits an attenuated linear rendering of the entire acoustic spectrum, in particular an attenuation of high frequencies, in particular beyond 8 kHz.

In other words, the device according to the invention tunes the sound, i.e. it takes the sound in its frequency content as it arrives at its entry 2, maintains this spectral content yet attenuating it, then equalizes is. The result, similar to a <<loudness>> effect is achieved by reducing the sound just like the ear would naturally do it, yet balancing the extreme frequencies, i.e. by intensifying the frequencies below 2 kHz and above 8 kHz.

To this end, at its entry 3, said device includes means 4 for attenuating and setting into resonance the sound. Preferably, said means 4 are comprised of a first portion of said channel 1 open on the outside at the level of the entry 3. The dimensions of this portion modify the attenuation or the setting into resonance of the perceived sound. Therefore, the length of the means 4 is determined according to the desired resonance. Similarly, the cross-section of the means 4 is determined according to the desired attenuation.

Preferably, but not restrictively, the means 4 permit to accentuate the high pitch before filtration.

This result can be achieved through a considerable and constant cross-section width until the filtration. In particular, said width should be related to its length, in order to maintain a ratio permitting to set into resonance and to keep in resonance the waves until the filtration.

As a non-restrictive example, shown in FIG. 3, the first portion of the channel 1 constituting the means 4 can have a circular internal cross-section of a diameter of 4 mm, whereas it has a length of 4.5 mm between the entry 2 and the filtration. In order to achieve a similar effect of resonances of frequencies, this embodiment uses a larger width with respect to its smaller length.

Compared to another embodiment, shown in FIG. 5, this cross-section can measure 1.4 mm, whereas the length is equal to 6.1 mm. As mentioned before, the cross-section is smaller on account of a more considerable length.

Anyway, the minimum cross-section of the entry 2 of the channel 1 should reach at least 0.5 mm, so as to pick up a wave with the largest acoustic spectrum possible, in particular without clipping the high frequencies. Compared to the state of the art, this more considerable entry width 2 diminishes the attenuation with respect to a smaller cross-section, in the range of a tenth of a millimeter, from 0.1 to 0.2 mm.

In general, the means 4 propagate the sound to the filtration means.

According to an embodiment, shown in FIG. 2, said means 4 open inside a housing 5 for receiving said filtration means in the form of at least one filter 6. Said housing 5 is in the form of a cavity having appropriate dimensions so as to, on the one hand, receive said filter 6 and, on the other hand, maintain the sound in resonance. In this case, the width of said housing 5 is equivalent to that of the filter 6, so as to maintain the latter within the cavity. The resonance is carried out, even in this case, according to the length of said housing 5, before and/or after the filter 6, but also along the latter.

As such, said filter 6 is chosen according to the desired acoustic characteristics. More specifically, according to a non-restrictive embodiment, the filter 6 has standard dimensions. It can be of any type, in particular of the type impedance acoustic resistance. Such a filter permits to obtain a regulated curve, in the manner of an <<acoustic damper>>.

Said filter, can be placed in front of the cavity, directly at the exit of the means 4, in the middle of the latter or at the back. The positioning of the filter 6 is studied so as to modify the sound according to the contemplated result.

In short, the housing 5 enclosing the filter 6 acts as a filtering resonator. It should be noted that the housing 5 enclosing the filter 6 can be aligned with the first portion of the channel 1, forming the attenuation means 4, offering continuity in maintaining the resonance of frequencies.

According to another embodiment, shown in FIG. 3, said filter 6 is placed at the end of the means 4, at the level of the exit of the latter, so that the sound opens directly onto said filter 6.

It can be observed in this case that the cross-section of the channel 1 has been widened optimally in order to improve the setting into resonance of the sound. In addition, at the exit 3 of said device, means 7 for accentuating the filtered sound are provided for. The latter are placed at the exit of the housing 5 and receive the filtered sound at its entry.

Said means 7 create a pavilioning effect generating a projection phenomenon. They accentuate more specifically the short-wave frequencies, such as the middle and high pitch. Therefore, said means 7 permit to intensify again the previously attenuated frequencies, contributing to the <<loudness>> effect and to the musicality of the sound at the exit 3.

To this end, said accentuation means 7 are comprised of a portion of said channel 1 having a frustoconical shape and the cross-section of which increases from said housing 5 toward said exit 3. The cross-section and the angle pf conicity of said accentuation means 7 are determined according to the desired accentuation of the sound.

In addition, the angle of conicity can vary according to the length. As mentioned before for the attenuation means 4, a larger length of the cone permits to achieve, with a smaller angle (FIG. 5), a result comparable to a larger angle for a smaller length (FIG. 3).

In the case shown in FIG. 3, the filter 6 is inserted directly at the entry of said accentuation means 7. For this reason, in this case, said filter 6 can be deprived of its protective cover 8, so that its maintenance is carried out along the walls of the accentuation means 7 through the edges 9 of its membrane 10, by pressing by force.

On the other hand, according to the contemplated embodiments, FIG. 2 or 3, the device according to the invention can be made of one single or two pieces. In fact, the insertion of the filter 6 in the housing 5 requires that the device be comprised of the two components cooperating by interlocking.

Whereas the configuration of FIG. 3 permits to directly insert said filter 6, through interlocking by force, maintained then by the stress of the walls of said accentuation means 7. It is then no longer necessary to have two components cooperating between them.

This invention also relates to an earplug, comprising an acoustic device as mentioned above. In particular, the plug in its entirety can be formed of said device. According to another embodiment, said device can be inserted, enclosed or framed by said plug.

Such a standard-dimension plug fits any type of ear. In addition, it can be formed of a shape-memory material, of the type expanded foam or similar, so as to be partially compressed, then restored its original shape, sticking in the ear of the user and attenuating the parasite sounds not filtered by the device according to the invention.

This invention also relates to a hearing aid, made by molding to measure. Adapted to the size of the user's ear, such a hearing aid stops any parasite sound because it perfectly matches the internal contours of said ear.

Such a hearing aid is formed so as to receive an acoustic device according to the invention and receives such a device. The latter can be removable or directly enveloped by the hearing aid during the molding stage.

More specifically, such a hearing aid can include, at the exit, a tube the cross-section and length of which are determined according to the audio result to be achieved. Said tube contributes to obtaining a more natural attenuated sound of tones perception through a phenomenon of reflection of middle and high pitch frequencies along said tube.

Therefore, this invention consists of a hearing protector capable of offering a linear attenuation of the filtered sound with a superior listening qualities and a more natural sound, through a combination of attenuation, then amplification of the filtered sound, set in resonance throughout the device.

The specific dimensioning, in particular through a larger width diminishing the attenuation effect, permits to cover a larger acoustic spectrum width, going up to the high frequencies, beyond 8 kHz and even up to 16 kHz.

Therefore, the invention permits to render said high frequencies perceived and filtered, giving a <<loudness>> effect to the sound at the exit 3.

The invention is, of course, not restricted to the examples illustrated and described above, which can have variants and modifications without therefore departing from the scope of the invention.

1-9. (canceled)

10. An acoustic device for linear perceived-sound attenuation, comprising a channel along which the sound propagates from an entry to an exit, wherein it comprises, combined so as to make the perceived sound undergo a linear attenuation, at its entry, means for attenuating and setting into resonance the sound propagating through the latter to at least one filter opening, at its exit, onto means for accentuating the filtered sound, in particular its short-wave frequencies, the accentuation means consisting of a portion of the channel having a frustoconical shape the cross-section of which increases from the housing toward the exit.

11. The acoustic device according to claim 10, wherein the attenuation means are formed of a portion of the channel over a length determined depending on the desired resonance.

12. The acoustic device according to claim 10, wherein the attenuation means are formed of a portion of the channel according to a cross-section determined depending on the desired attenuation.

13. The acoustic device according to claim 10, wherein the means open inside a housing for receiving the filter, the housing being in the form of a cavity having appropriate dimensions so as to, on the one hand, receive the filter and, on the other hand, maintain the sound in resonance.

14. The acoustic device according to claim 10, wherein the length, the cross-section and the angle of conicity of the accentuation means are determined depending on the desired accentuation.

15. The acoustic device according to claim 10, wherein the entry cross-section has a diameter of at least 0.5 millimeters.

16. An earplug, comprising an acoustic device according to claim 10.

17. An hearing aid, made by molding to measure and formed so as to receive, and receiving an acoustic device according to claim 10.

18. The hearing aid according to claim 17, comprising, at the exit, a tube the cross-section and length of which are determined depending on the audio result to be achieved.
